(19) United States
(12) Patent Application Publication
(10) Pub. No.: US 2008/0213275 A1
(43) Pub. Date: Sep. 4, 2008

(54) USE OF MTKI 1 FOR TREATING OR PREVENTING BONE CANCER

(76) Inventors: Eddy Jean Edgard FREYNE, Rumst (BE); Michel Marie Francois Janicot, Mol (BE); Theo Frans Meert, Boom (BE); Timothy Pietro Suren Perera, Geel (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/875,429

(22) Filed: Oct. 19, 2007

Related U.S. Application Data
(60) Provisional application No. 60/863,167, filed on Oct. 27, 2006.
(60) Provisional application No. 60/976,188, filed on Sep. 28, 2007.

(51) Int. Cl. A61K 39/395; A61K 31/519; A61K 31/4985; A61P 35/00; A61K 31/704

(52) U.S. Cl. 424/141.1; 514/257; 514/34; 514/249

(57) ABSTRACT

The present invention is concerned with the finding that the macrocyclic quinazoline derivative 4,6-ethanediylidenepyrimido[4,5-b][6,1,12]benzoxadiazacyclo-pentadecine, 17-bromo-8,9,10,11,12,13,14,19-octahydro-20-methoxy-13-methyl-, described as compound 22 in PCT publication WO2004/105765, is useful in the manufacture of a medicament for the treatment or prevention of bone cancers and methods for killing bone cancer cells, including osteosarcomas, chondrosarcomas, myeloma bone disease and osteolytic bone metastases from other primary sites. It accordingly provides methods for treating, preventing, delaying or mitigating bone cancer, or for preventing and treating of bone loss associated with cancer metastases.

### CROSS-REFERENCE TO RELATED APPLICATION

This application claims priority to U.S. Provisional Application for Patent No. 60/863,167, filed Oct. 27, 2006, and U.S. Provisional Application for Patent No. 60/976,188, filed Sep. 28, 2007, the entire disclosures of which are hereby incorporated in their entirely.

### FIELD OF THE INVENTION

The present invention is concerned with the finding that the macrocyclic quinazoline derivative 4,6-ethanediylidenepyrimido[4,5-b][6,1,12]benzoxadiazacyclo-pentadecine, 17-bromo-8,9,10,11,12,13,14,19-octahydro-20-methoxy-13-methyl, described as compound 22 in PCT publication WO2004/105765, is useful in the manufacture of a medicament for the treatment or prevention of bone cancers and methods for killing bone cancer cells, including osteosarcomas, chondrosarcomas, myeloma bone disease and osteolytic bone metastases from other primary sites. It accordingly provides methods for treating, preventing, delaying or mitigating bone cancer, or for preventing and treating of bone loss associated with cancer metastases.

### BACKGROUND OF THE INVENTION

“Bone cancer” includes primary bone cancer cells such as osteosarcoma cells, cells from Ewing's family of tumors, chondrosarcoma cells, malignant giant cell tumor cells, malignant fibrous histiocytoma cells and adamantinoma cells, as well as secondary bone cancer cells that have metastasized from other tissues, including breast, lung, prostate and kidney.

Osteosarcoma is a malignant tumor of bone, which is most prevalent in adolescents and young adults. Osteosarcoma accounts for approximately 5% of the tumors in childhood and 80% of these tumors originate around the knee. The prognosis is often poor and within 1 year after commencing definitive therapy, about 30% of patients diagnosed with osteosarcoma will develop lung metastasis. The prognosis appears to be determined by the site of metastases and surgical resectability of the metastatic disease, either at diagnosis or following a variable period of chemotherapy. Patients who have complete surgical ablation of the primary and metastatic tumor (when confined to the lung) following chemotherapy may attain long-term survival, although event-free survival remains about 20% for patients with metastatic disease at diagnosis. Patients developing recurrent disease often have a poor prognosis and die within 1 year of the development of metastatic disease.

Chemotherapy is often ineffective, resulting in a high mortality rate. Hence, it is important that new therapeutic approaches are evaluated for this malignant disease.

Myeloma bone disease is a cancer of antibody-producing plasma cells in the bone marrow. Proliferation of the cancerous plasma cells, referred to as myeloma cells, causes a variety of effects, including lytic lesions (holes) in the bone, decreased red blood cell number, production of abnormal proteins (with attendant damage to the kidney, nerves, and other organs), reduced immune system function, and elevated blood calcium levels (hypercalcemia).

When myeloma cells are present at distinct skeletal locations, the disease is referred to as multiple myeloma.

Although responsible for only 1% of all cancers in the United States, with 14,600 new cases reported in 2002, myeloma is the second most common blood cancer and may be increasing in prevalence, particularly among individuals under age 55 (International Myeloma Foundation). Many different treatment options are available or in development, but there is neither a cure nor agreement on an optimal myeloma management regimen. Patients are treated with chemotherapy as well as symptom-specific treatments for one or more of hypercalcemia, increased infection risk, kidney failure, anemia, hyperviscosity of blood, elevated stroke risk, bone destruction and pain, and muscle weakness. Unfortunately, dramatic reduction in the number of myeloma cells does not necessarily translate into longer remissions or survival times, and therapies that were effective before a remission may not prove effective upon relapse of the disease.

One of the most prevalent and significant characteristics of myeloma is the activation of osteoclasts, multinucleated cells that absorb bone, leading to bone thinning, lytic bone lesions, and bone fracture. Lytic bone lesions occur in 70-80% of multiple myeloma patients and are frequently associated with severe bone pain and pathologic fractures. In normal bone functioning, a balance exists between osteoclasts, which resorb bone, and osteoblasts, cells that produce bone. This balance is upset in myeloma patients, and more bone is resorbed than produced. The increased osteoclastic bone resorption occurs adjacent to the myeloma cells and not in areas of normal bone marrow, indicating that the osteoclast activation occurs by a local mechanism. Although it is well accepted that myeloma cells activate osteoclasts, the precise mechanism by which this occurs is unknown. Myeloma cells, in culture, produce or induce production of several osteoclast-activating factors (OAFs) whose specific roles in vivo are yet to be determined. Recently, the chemokine macrophage inflammatory protein-1a (MIP-1a) has been implicated in osteoclast activation in vitro (S. J. Choi et al, Blood 96: 671-675 (2000)). Therapies addressing mechanisms involving OAFs are presently under development.

Currently, bone indications of multiple myeloma are treated primarily with bisphosphonates, a class of chemicals that inhibits osteoclast activity or osteoclast attachment to bone surface and eventually leads to osteoclast cell death. They may also affect myeloma cells directly. Bisphosphonates are administered by infusion. Third-generation bisphosphonates are currently under development, but even improved versions of the drugs may have potential side effects including hypocalcemia, kidney damage, and increased pain.

Bisphosphonates do not completely block the bone destruction process, and patients eventually develop new bone lesions. An alternative therapy for bone destruction in multiple myeloma that can be administered orally would be highly beneficial.

Bone metastases are often associated with advanced cancer and are most common with breast, prostate and thyroid carcinomas and multiple myeloma (supra). Bone metastases are present in 65-75% of patients with advanced (metastatic) breast cancer. Metastatic bone lesions may be lytic or sclerotic in nature depending upon whether increased osteoclastic or osteoblastic activity predominates; if both processes are equally active, they are termed mixed lesions. Bone metastases in breast cancer patients usually involve osteolytic disease, where normal bone homeostasis is disrupted and skewed towards excessive resorption of bone (Coleman R E, Cancer Treat Rev. 27(3), 165-76 (2001)). Tumor-induced skeletal damage is mediated by osteoclasts that are stimulated directly or indirectly to dissolve bone by local factors (e.g. prostaglandin E, interleukin-1, tumor necrosis factor and procathepsin D) released by tumor cells or associated immune cells, or by systemic factors, such as parathyroid hormone-related peptide. The most frequently affected skeletal sites are the vertebrae, pelvis, ribs, femur and skull.

Patients with bone metastases experience considerable morbidity, including bone pain, pathological fractures, hypercalcaemia, reduced mobility and spinal cord or nerve root compression. Despite the importance of these clinical problems, there are few available treatments for bone loss associated with cancer metastasis. Thus, there remains a need in the art to identify new agents and methods for preventing or treating cancer metastasis, including osteolytic bone metastases.

### SUMMARY OF THE INVENTION

The invention is directed in part to methods of treating or preventing bone cancer, and to methods of treating or preventing bone loss associated with cancer metastases, utilizing certain compounds described in WO 2004/105765, the disclosure of which is hereby incorporated by reference in its entirety.

In one embodiment, the present invention provides the use of the macrocyclic quinazoline derivative 4,6-ethanediylidenepyrimido[4,5-b][6,1,12]benzoxadiazacyclo-pentadecine, 17-bromo-8,9,10,11,12,13,14,19-octahydro-20-methoxy-13-methyl, described as compound 22 in PCT publication WO2004/105765, in the manufacture of a medicament for the treatment or prevention of bone cancers and methods for killing bone cancer cells, including osteosarcomas, chondrosarcomas, myeloma bone disease and osteolytic bone metastases. It accordingly provides methods for treating, preventing, delaying or mitigating bone cancer, or for preventing and treating of bone loss associated with cancer metastases.

In related embodiment, the invention provides a method of inhibiting metastatic spread of a cancer to skeletal system, in a mammalian subject comprising administering to a mammalian subject suspected of having metastatic cancer a compound of the invention, in an amount effective to inhibit metastatic spread of the cancer to the skeletal system; and a method for treating bone cancer comprising administering to a mammalian subject diagnosed with a cancer a composition comprising a compound of the invention, in an amount effect to reduce growth or neoplastic spread of the bone cancer. It will be appreciated that any reduction in the rate of cancer growth or spread (which can prolong life and quality of life) is indicative of successful treatment. In preferred embodiments, cancer growth is halted completely. In still more preferred embodiments, cancers shrink or are eradicated entirely. Preferred subjects for treatment are human subjects, but other animals, especially murine, rat, bovine, porcine, primate, and other model systems for cancer treatment, are contemplated. Metastatic cancers as used herein are contemplated to include a variety of cancers can metastasize to the bone, but the most common metastasizing cancers are breast, lung, renal, multiple myeloma, thyroid and prostate. By way of example, other cancers that have the potential to metastasize to bone include but are not limited to adenocarcinoma, blood cell malignancies, including leukemia and lymphoma; head and neck cancers; gastrointestinal cancers, including stomach cancer, colon cancer, colorectal cancer, pancreatic cancer, liver cancer; malignancies of the female genital tract, including ovarian carcinoma, uterine endometrial cancers and cervical cancer; bladder cancer; brain cancer, including neuroblastoma; sarcoma, osteosarcoma; and skin cancer, including malignant melanoma and squamous cell cancer. The present invention especially contemplates prevention and treatment of tumor-induced osteolytic lesions in bone

In one variation of the foregoing methods of treatment, the compounds are administered along with a second cancer therapeutic agent. The second agent can be any chemotherapeutic agent, radioactive agent, radiation, nucleic acid encoding a cancer therapeutic agent, antibody, protein, and/or other anti-lymphangiogenic agent or an anti-angiogenic agent. The second agent may be administered before, after, or concurrently with the compounds of the invention.

In one variation, the subject to be treated has been diagnosed with an operable tumor, and the administering step is performed before, during, or after the tumor is resected from the subject. Compound treatment in conjunction with tumor resection is intended to reduce or eliminate regrowth of tumors from cancer cells that fail to be resected.

Stated more generically, the invention provides a method of treating or preventing bone cancer, and to methods of treating or preventing bone loss associated with cancer metastases comprising the step of administering to a mammal (including, but not limited to humans, rats, canines, bovines, porcines, and primates) in need thereof a compound of the invention.

### BRIEF DESCRIPTION OF THE DRAWINGS

FIG. 1: Effect of treatment on spontaneous lifting behaviour of the left hind paw. The data is presented as the percentage of the time the paw was raised over an observation period of 4 minutes. The vehicle group was dosed by oral gavage, daily with a 20% HPCD solution at pH 4.0. Compound 1 was dosed once daily at its maximum tolerated dose (MTD) of 200 mg per kg, Iressa was also dosed at its maximum tolerated dose, by oral gavage, of 50 mg per kg daily for 14 days.

FIG. 2: Representative reconstructions from μCt s of the ipsilateral left hindlimbs showing osteolytic bone destruction in the tumor inoculated animals.

FIG. 3: Dose dependent inhibition of breast tumor growth in a bone cancer metastasis model. MDA B231

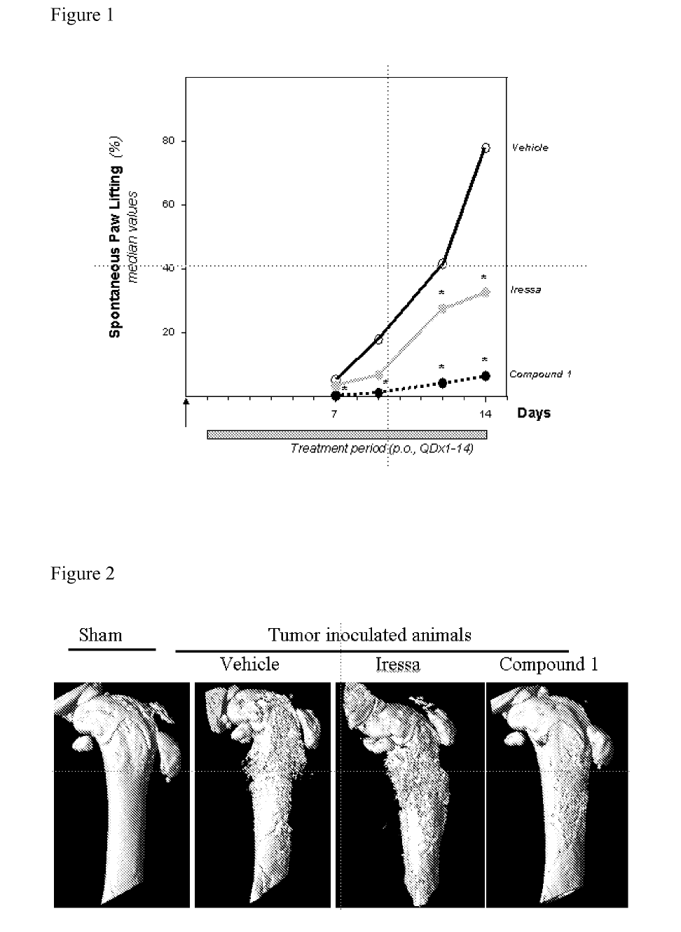

### DETAILED DESCRIPTION OF THE INVENTION

WO-2004/105765 describes the preparation, formulation and pharmaceutical properties of macrocyclic quinazoline derivatives of formula (I) as multi targeted kinase inhibitors (MTKIs).

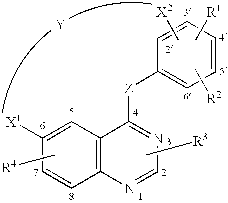

It has now been found that one compound in the aforementioned class, i.e 4,6-ethanediylidenepyrimido[4,5-b][6,1,12]benzoxadiazacyclo-pentadecine, 17-bromo-8,9,10,11,12,13,14,19-octahydro-20-methoxy-13-methyl, described as compound 22 in the aforementioned PCT publication, herein also referred to as MTKI 1 or Compound 1, has clinical activity in bone cancer models and accordingly provide the use of these compounds for the preparation of a pharmaceutical composition for treating bone cancer, including primary bone cancers and bone metastases as defined hereinbefore.

The present invention also concerns a method of treating tumor-induced osteolytic lesions in bone of a mammal, comprising the step of administering a therapeutically effective amount of a compound according to the invention to said mammal.

Accordingly, in one aspect the present invention provides the use of 4,6-ethanediylidenepyrimido[4,5-b][6,1,12]benzoxadiazacyclo-pentadecine, 17-bromo-8,9,10,11,12,13,14,19-octahydro-20-methoxy-13-methyl or a pharmaceutically acceptable acid or base addition salt thereof, in the manufacture of a medicament for the treatment or prevention of bone cancer, including primary bone cancers and bone metastases as defined hereinbefore.

A further aspect of the present invention is directed to a method for the treatment of prevention of bone cancer in a mammalian subject, comprising administering a therapeutically effective amount of 4,6-ethanediylidenepyrimido[4,5-b][6,1,12]benzoxadiazacyclo-pentadecine, 17-bromo-8,9,10,11,12,13,14,19-octahydro-20-methoxy-13-methyl or a pharmaceutically acceptable acid or base addition salt thereof, to a mammalian subject in need of such treatment.

The pharmaceutically acceptable acid or base addition salts as mentioned hereinabove are meant to comprise the therapeutically active non-toxic acid and non-toxic base addition salt forms which MTKI 1 is able to form. The basic properties can be converted in their pharmaceutically acceptable acid addition salts by treating said base form with an appropriate acid. Appropriate acids comprise, for example, inorganic acids such as hydrohalic acids, e.g. hydrochloric or hydrobromic acid; sulfuric; nitric; phosphoric and the like acids; or organic acids such as, for example, acetic, propanoic, hydroxyacetic, lactic, pyruvic, oxalic, malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids.

The acidic properties may be converted in their pharmaceutically acceptable base addition salts by treating said acid form with a suitable organic or inorganic base. Appropriate base salt forms comprise, for example, the ammonium salts, the alkali and earth alkaline metal salts, e.g. the lithium, sodium, potassium, magnesium, calcium salts and the like, salts with organic bases, e.g., the benzathine, N-methyl-D-glucamine, hydrabamine salts, and salts with amino acids such as, for example, arginine, lysine and the like.

The terms acid or base addition salt also comprise the hydrates and the solvent addition forms which MTKI 1 is able to form. Examples of such forms are e.g., hydrates, alcoholates and the like.

In particular, the present invention is concerned with a use of the dihydrobromide salt of 4,6-ethanediylidenepyrimido[4,5-b][6,1,12]benzoxadiazacyclo-pentadecine, 17-bromo-8,9,10,11,12,13,14,19-octahydro-20-methoxy-13-methyl, i.e., 17-bromo-8,9,10,11,12,13,14,19-octahydro-20-methoxy-13-methyl-4,6-ethanediylidenepyrimido [4,5-b][6,1,12]benzoxadiazacyclopentadecine dihydrobromide, in any of the aforementioned uses for MTKI 1.

In a further embodiment, the present invention provides the use of the aforementioned MTKI 1 for the preparation of a pharmaceutical composition for the prevention and/or treatment of bone cancers.

The present invention also concerns a method of preventing and/or treating bone cancer in a mammal, comprising the step of administering a therapeutically effective amount of the aforementioned MTKI 1 to said mammal.

In a further embodiment, the present invention provides the use of MTKI 1 for the preparation of a pharmaceutical composition for the prevention and/or treatment of bone loss.

The present invention also concerns a method for preventing and/or treating of bone loss associated with cancer metastases in a mammal, comprising the step of administering a therapeutically effective amount of MTKI 1 to said mammal.

Accordingly, in a further aspect, the most preferred compounds for use in accordance with the present invention are those selected from the group consisting of compounds having the following structure:

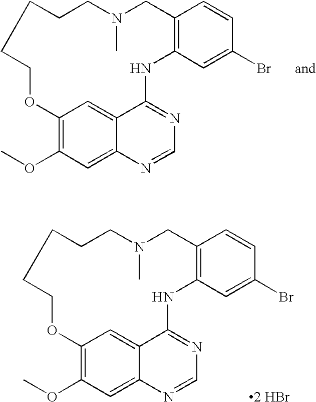

The compounds according to the invention can be prepared and formulated into pharmaceutical compositions by methods known in the art and in particular according to the methods described in the published patent specification WO-2004/105765 mentioned herein and incorporated by reference.

A suitable preparation of the preferred compound used in this invention, taken from WO-2004/105765, follows:

### EXAMPLE 1

a) Preparation of 1-pentanol, 5-[[(4-bromo-2-nitrophenyl)methyl]amino]- (intermediate 1)

A solution of 4-bromo-2-nitro- benzaldehyde,(0.013 mol), 5-amino-1-pentanol (0.013 mol) and titanium, tetrakis (2-propanolato) (0.014 mol) in EtOH (15 ml) was stirred at RT for 1 hour, then the reaction mixture was heated to 50° C. and stirred for 30 min. The mixture was cooled to RT and NaBH₄ (0.013 mol) was added portionwise. The reaction mixture was stirred overnight and then poured out into ice water (50 ml). The resulting mixture was stirred for 20 min., the formed precipitate was filtered off (giving Filtrate (I)), washed with H₂O and stirred in DCM (to dissolve the product and to remove it from the Ti-salt). The mixture was filtered and then the filtrate was dried (MgSO₄) and filtered, finally the solvent was evaporated. Filtrate (I) was evaporated until EtOH was removed and the aqueous concentrate was extracted 2 times with DCM. The organic layer was separated, dried (MgSO₄), filtered off and the solvent was evaporated, yielding 3.8 g (93%) of intermediate 1.

### EXAMPLE 2

a) Preparation of 1-pentanol, 5-[[(4-bromo-2-nitrophenyl)methyl]methylamino]-(intermediate 2)

A solution of intermediate 50 (0.0047 mol), formaldehyde (0.025 mol) and titanium, tetrakis (2-propanolato) (0.0051 mol) in EtOH (150 ml) was heated to 50° C. and stirred for 1 hour, then NaBH₄ (0.026 mol) was added portionwise at RT. The reaction mixture was stirred overnight and then quenched with water (100 ml). The resulting mixture was stirred for 1 hour; the formed precipitate was filtered off and washed. The organic filtrate was concentrated, then the aqueous concentrate was extracted with DCM and dried. The solvent was evaporated and the residue was filtered over silica gel (eluent: DCM/CH₃OH from 98/2 to 95/5). The product fractions were collected and the solvent was evaporated, yielding 0.5 g of intermediate 2.

b) Preparation of 1-pentanol, 5-[[(4-bromo-2-nitrophenyl)methyl]methylamino]-, acetate (ester) (intermediate 3)

A solution of intermediate 2 (0.0015 mol) and pyridine (0.015 mol) in acetic anhydride (8 ml) was stirred overnight at RT, then the solvent was evaporated and co-evaporated with toluene, yielding intermediate 3.

c) Preparation of 1-pentanol, 5-[[(2-amino-4-bromophenyl)methyl]methylamino]-, acetate (ester) (intermediate 4)

A mixture of intermediate 3 (0.0015 mol) in THF (50 ml) was hydrogenated with Pt/C 5% (0.5 g) as a catalyst in the presence of thiophene solution (0.5 ml) [H179-034]. After uptake of H₂ (3 equiv.), the catalyst was filtered off and the filtrate was evaporated, yielding 0.5 g of intermediate 4.

d) Preparation of 6-quinazolinol, 4- [[2-[[[5-(acetyloxy)pentyl]methylamino]methyl]-5-bromophenyl]amino]-7-methoxy-, acetate (ester) (intermediate 5)

A mixture of intermediate 4 (0.0015 mol) and 4-chloro-7-methoxy-6-quinazolinol acetate (ester) (0.0015 mol) in 2-propanol (30 ml) was heated to 80° C. and the reaction mixture was stirred for 1 day. The solvent was evaporated under reduced pressure and the residue was used as such in the next reaction step, yielding 0.83 g of intermediate 5.

e) Preparation of 6-quinazolinol, 4-[[5-bromo-2-[[(5-hydroxypentyl)methylamino]methyl]phenyl]amino]-7-methoxy-(intermediate 6)

A solution of intermediate 5 (0.0015 mol) in methanol (25 ml) was stirred at RT and a solution of K₂CO₃ (0.003 mol) in H₂O (2.5 ml) was added, then the reaction mixture was heated to 60° C. and stirred for 18 hours. The solvent was evaporated and H₂O (20 ml) was added, then the mixture was neutralized with acetic acid and the formed precipitate was filtered off. The filtrate was concentrated under reduced pressure and the concentrate was extracted with DCM, filtered, then dried (MgSO₄) and the mixture was concentrated under reduced pressure, yielding 0.5 g (70%) of intermediate 6.

### EXAMPLE 3

a)Preparation of 4,6-ethanediylidenepyrimido[4,5-b][6,1,12]benzoxadiazacyclo-pentadecine, 17-bromo-8,9,10,11,12,13,14,19-octahydro-20-methoxy-13-methyl-(compound MTKI1)

A solution of intermediate 6 (0.0011 mol) in THF (50 ml) was stirred at RT and tributylphosphine (0.0016 mol) was added, then 1,1′-(azodicarbonyl)bis-piperidine (0.0016 mol) was added and the reaction mixture was stirred for 2 hours. The solvent was evaporated until ⅓ of the initial volume. The resulting precipitate was filtered off and washed. The filtrate was evaporated and the residue was purified by RP high-performance liquid chromatography. The product fractions were collected and the organic solvent was evaporated. The aqueous concentrate was extracted 2 times with DCM and the organic layer was dried (MgSO₄), then filtered off. The solvent was evaporated and the residue was dried (vac.) at 50° C., yielding 0.004 g (0.8%) of compound MTKI1.

To prepare the aforementioned pharmaceutical compositions, a therapeutically effective amount of the particular compound, optionally in addition salt form, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for systemic administration such as oral, percutaneous, or parenteral administration; or topical administration such as via inhalation, a nose spray, eye drops or via a cream, gel, shampoo or the like. For example, in preparing the compositions in oral dosage form, any of the usual pharmaceutical media may be employed, such as, for example, water, glycols, oils, alcohols and the like in the case of oral liquid preparations such as suspensions (including nanosuspensions), syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable solutions containing compounds of formula (I) may be formulated in an oil for prolonged action. Appropriate oils for this purpose are, for example, peanut oil, sesame oil, cottonseed oil, corn oil, soy bean oil, synthetic glycerol esters of long chain fatty acids and mixtures of these and other oils. Injectable suspensions may also be prepared in which case appropriate liquid carriers, suspending agents and the like may be employed. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g., as a transdermal patch, as a spot-on or as an ointment. As appropriate compositions for topical application there may be cited all compositions usually employed for topically administering drugs e.g. creams, gels, dressings, shampoos, tinctures, pastes, ointments, salves, powders and the like. Application of said compositions may be by aerosol, e.g. with a propellent such as nitrogen, carbon dioxide, a freon, or without a propellent such as a pump spray, drops, lotions, or a semisolid such as a thickened composition which can be applied by a swab. In particular, semisolid compositions such as salves, creams, gels, ointments and the like will conveniently be used.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

Preferably, a therapeutically effective amount of the pharmaceutical composition comprising a compound according to the invention, is administered orally or parenterally. Said therapeutically effective amount is the amount that effectively prevents metastasis and/or growth or reduces the size of a variety of neoplastic disorders or cell proliferative disorders (supra) in patients. On the basis of the current data, it appears that a pharmaceutical composition comprising a compound of the present invention, and in particular 4,6-ethanediylidenepyrimido[4,5-b][6,1,12]benzoxadiazacyclo-pentadecine, 17-bromo-8,9,10,11,12,13,14,19-octahydro-20-methoxy-13-methyl (MTKI1, or Compound 1) as the active ingredient can be administered orally in an amount of from 10 mg to several (1 to 5) grams daily, either as a single dose or subdivided into more than one dose, including, e.g., two, three or even four times daily. A preferred amount ranges from 500 to 4,000 mg daily. A particularly, preferred dosage for such a compound is in the range of 750 mg to 3,000 mg daily. It will be appreciated that the amount of a compound according to the present invention, also referred to here as the active ingredient, which is required to achieve a therapeutic effect will, of course, vary with, the route of administration, the age and condition of the recipient, and the particular disorder or disease being treated. The optimum dosage amounts and regimen can be readily determined by those skilled in the art using conventional methods and in view of the information set out herein. This treatment can be given either continuously or intermittently, including, e.g., but not limited to, cycles of 3-4 weeks with treatment given for 1-21 days per cycle or other schedules shown to be efficacious and safe.

One illustrative formulation is as follows:

### EXAMPLE 4

### Formulation:

The product MTKI1 can be prepared as a 10-mg/mL oral solution, pH 2. It contains an excipient, Captisol® (chemical name: sulfobutyl ether-β-cyclodextrin, SBE-β-CD), citric acid, Tween® 20, HCl, and NaOH in purified water. The formulation can be stored refrigerated (2-8° C.; 36-46° F.) and allowed to warm to room temperature for maximally 1 hour prior to dose preparation.

The product MTKI1 can also be prepared as 50-mg, 100-mg and 300-mg oral immediate release capsules, containing the active chemical entity MTKI1, lactose monohydrate (200 mesh), sodium lauryl sulphate and magnesium stearate in hard gelatin capsules, sizes 3, 4 and 00, respectively. The capsules may also contain any or all of the following ingredients: gelatin, red iron oxide and titanium oxide.

The above MTKI 1 may be used in combination with one or more other cancer treatments. Such combinations could encompass any established antitumor therapy, such as, but not limited to, chemotherapies, irradiation, and target based therapies such as antibodies and small molecules (such as for example bisphosphonates, taxanes, anthracyclines, capecitabine, Herceptin, docetaxel, satraplatin, cetuximab, avastin, aromatase inhibitors and methothrexate). These therapies may be combined in systemic therapy, or local instillation/administration (e.g. intrathecally), depending on optimum efficacy/safety requirements.

The MTKI 1 and the further anti-cancer agent may be administered simultaneously (e.g. in separate or unitary compositions) or sequentially in either order. In the latter case, the two compounds will be administered within a period and in an amount and manner that is sufficient to ensure that an advantageous or synergistic effect is achieved. It will be appreciated that the preferred method and order of administration and the respective dosage amounts and regimens for each component of the combination will depend on the particular MTKI and further anti-cancer agents being administered, their route of administration, the particular tumor being treated and the particular host being treated. The optimum method and order of administration and the dosage amounts and regimen can be readily determined by those skilled in the art using conventional methods and in view of the information set out herein.

Experimental Data

The unique physico-chemical properties of MTKI 1, also referred to herein as Compound 1, has resulted in an extremely favourable tissue distribution profile including the ability to cross the intact blood brain barrier whilst still retaining good cellular activity and oral bioavailability. Here, we further demonstrate that this preferential tissue distribution to the bone marrow compartment results in significant anti tumoral activity using experimental models of bone metastases.

NCTC2472 fibrosarcoma (ATCC Rockvile, Md. USA) or MDA-MB231 breast cancer cells (Dr. Yoneda, Univ. of Michigan, USA) (bone homing variant) were injected into the tibia of nude mice, the hole sealed and tumor growth observed at predefined times (Vermeirsch, H et al Pharmacol Biochem Behav. 2004 October; 79(2):243-51). Spontaneous ‘paw lifting’ was used as a pain response indicator whilst μCt and histology was use to demonstrate osteolytic anti tumor activity and tumor growth.

Methods

Animal Model

Male C3H/HeNCrl mice for the NCTC2472 mouse fibrosarcoma cells (20-25 g, Charles River, Sulzfeld, Germany) or female NMRI Nude mice for the MDA MB 231 human breast cancer cells (Janvier, France) were used. Induction of bone cancer was carried out as previously described (Schwei et al., 1999¹). Induction of general anaesthesia was performed under 4% isoflurane in a mixture of 30% O₂ and 70% air (1000 ml/ min). Anaesthesia was then maintained at 2.5% isoflurane for the duration of the surgical procedure. The left hind paw was shaved and disinfected with povidone-iodine followed by 70% ethanol. A superficial 1 cm incision was made over the knee overlaying the patella. The patella ligament was cut, exposing the condyles of the distal femur. A 23-gauge needle was inserted at the level of the intercondylar notch and the intramedullary canal of the femur to create a cavity for injection of the cells. Tumour cells (2.5×10⁶ cells/20 μl) were then injected into the bone cavity using a 0.3 ml syringe. To prevent leakage of cells outside the bone, the injection site was sealed with dental acrylic (Paladur, Heraeus Kulzer, GmbH, Wehrheim, Germany) and the wound closed with skin stitches. For the sham-operated group, an identical procedure was followed except that medium without cells was injected.

Drug Treatment:

Treatment was initiated on day 1 following tumor cell induction. Mice were treated once daily (Q1D) with either vehicle (20% Hydroxypropyl-β-cyclodextrine, pH 4.0) or vehicle formulated to give a dose of 200 or 50 mg/kg of Compound 1 respectively by gavage (p.o.) administered in a volume of 10 ml/kg body weight. Mice were treated up to 18 days after bone tumor induction.

Pain Assessment:

Pain behaviours (see below) were evaluated in the group of sham and bone tumour mice and were behaviourally tested during a 2-week period prior to and 7, 9, 12 and 14 days after tumour inoculation. At the end of the experiment the femur of the left hind limb was sampled and used for pCT scanning as described in Vermeirsh et al., (2004)². Spontaneous lifting behavior: Animals were habituated to the laboratory room at least 30 minutes in a transparent acrylic cylinder of 20 cm diameter and thereafter observed during 4 minutes for spontaneous lifting behaviour of the left hind paw.

Evaluation of Bone Destruction:

Bone analysis was carried out on ipsilateral left hind limbs prior to and 7, 12, 15 and 18 days following cell injection. Limbs were fixed in 10% phosphate-buffered formalin and transferred to a plastic cuvette filled with 70% ethanol for scanning using the SkyScan microtomograph (Skyscan 1067®, Skyscan, Aartselaar, Belgium). For medium resolution measurement, the X-ray beam was collimated to a diameter of 18 mm, line spacing and point resolution were set at 0.254 and 0.127 mm, respectively. After standardized reconstruction, the datasets for each bone were re-sampled using computer software (Ant, 3D-creator vs. 2.2e, Skyscan, Aartselaar, Belgium) so that the medial axis of the bone was centrally oriented for each bone. Scans were processed and a two- and three-dimensional morphometric analysis was performed on a 5 mm femur bone segment at proximal end of the patellar trochlea using free software (CTanalyzer vs. 1.02, Skyscan, Aartselaar, Belgium).
1. Schwei M J, Honore P, Rogers S D, Salak-Johnson J L, Finke M P, Ramnaraine M L, et al. Neurochemical and cellular reorganization of the spinal cord in a murine model of bone cancer pain. J Neurosci 1999; 19:10886-97.
2. Vermeirsch, H., Nuydens, R., Salmon, P. L., and Meert, T. F. Pharmacol. Biochem. Behav. 2004. 79: 243-251.

Results

Pain Assessment

Animals were habituated to the laboratory room at least 30 minutes in a transparent acrylic cylinder of 20 cm diameter and thereafter observed during 4 minutes for spontaneous lifting behaviour of the left hind paw. The data is presented as the percentage of the time the paw was raised over this period of time. The vehicle group was dosed by oral gavage, daily with a 20% HPCD solution at pH 4.0. Compound 1 was dosed once daily at its maximum tolerated dose (MTD) of 200 mg per kg, Iressa was also dosed at its maximum tolerated dose, by oral gavage, of 50 mg per kg daily for 14 days. The vehicle treated group of animals displayed detectable paw lifting behaviours starting seven days post tumor cell inoculation. The percentage of the time the animals paws were raised during the observation period increased at both day 9, 12 and 14 post inoculation at which time the paw was raised 80% of the time. Dosing animals with Compound 1 at its MTD was found to reduce the time the animals did not use their left paws quite dramatically so that at day 14, spontaneous paw lifting behaviour was only detected to occur ˜8% of the time. The reference compound used in this study, Iressa when dosed at its MTD also led to a statistically significant reduction in spontaneous paw lifting behaviour, however the effect was far less extensive with spontaneous paw lifting being observed more than 35% of the time.

Evaluation of Bone Destruction

Representative reconstructions from μCt s of the ipsilateral left hindlimbs showing osteolytic bone destruction in the tumor inoculated animals (FIG. 2). Considerable bone loss was observed in the vehicle and Iressa treated groups whilst significantly less bone destruction can be seen in the Compound 1 treated animals. The sham operated animals showed no signs of osteolytic activity.

Animal Model

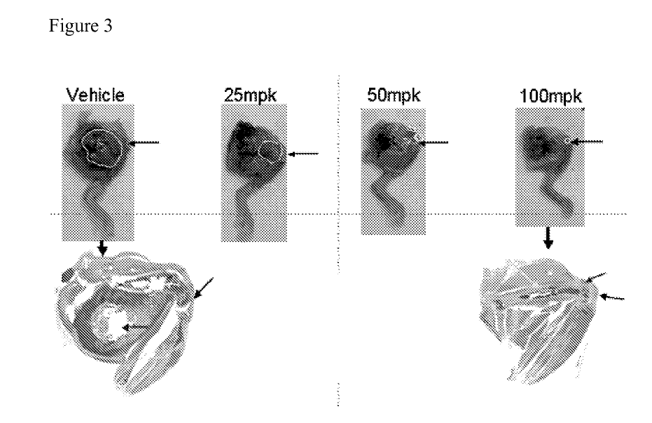

In the bone cancer metastasis model MDA B231 bone homing clone cells were inoculated into the tibia as described. After 42 days, animals were sacrificed and the amputated paws placed in fixative. The legs were de-calcified and sections cut to determine levels of bone destruction. The vehicle treated animals were observed to have large tumor mass (encircled area in FIG. 3) that has expanded out of the initial site of inoculation (black arrow in FIG. 3) and in the process resulted in significant destruction of the bone (See FIG. 2). The amount of tumor growth and the amount of bone destruction was seen to be dose dependently reduced with the highest dose of Compound 1 tested in this study, 100 mg per kg, po, qd, showing no signs of tumor cell not any signs bone destruction. The latter is also apparent from the histological sections in FIG. 3. In vehicle treated animals a large tumor mass has extended into the femur (grey arrow), where in animals treated with 100 mg per kg, po, qd, of MTKI 1, detectable tumor is hardly present (grey arrow) and close to the site of inoculation (black arrow).

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

1. A method of treating or preventing bone cancer or bone cancer metastases in a mammal, said method comprising administering a therapeutically effective amount of a compound chosen from the group consisting of 4,6-ethanediylidenepyrimido[4,5-b][6,1,12]benzoxadiazacyclo-pentadecine, 17-bromo-8,9,10,11,12,13,14,19-octahydro-20-methoxy-13-methyl- or a pharmaceutically acceptable acid or base addition salt thereof, or 17-bromo-8,9,10,11,12,13,14,19-octahydro-20-methoxy-13-methyl-4,6-ethanediylidenepyrimido [4,5-b][6,1,12]benzoxadiazacyclopentadecine dihydrobromide, to a mammal in need of such treatment.

2. A method of claim 1 in which the compound has the following structure:

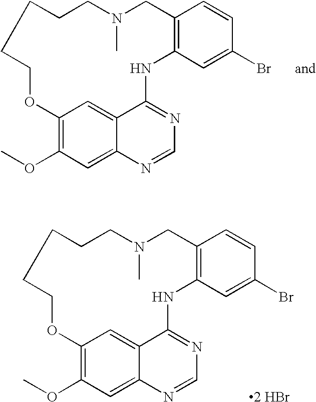

3. The method as claimed in claim 1, wherein a therapeutically effective amount of the compound is administered orally or parenterally.

4. The method as claimed in claim 1 wherein 4,6-ethanediylidenepyrimido[4,5-b][6,1,12]benzoxadiazacyclo-pentadecine, 17-bromo-8,9,10,11,12,13,14,19-octahydro-20-methoxy-13-methyl- or a pharmaceutically acceptable acid or base addition salt thereof, is administered in combination with a further anti-cancer agent.

5. A method according to claim 1 wherein the further anti-cancer agent is selected from the group consisting of bisphosphonates, radiation, taxanes, anthracyclines, capecitabine, Herceptin, docetaxel, satraplatin, cetuximab, avastin, aromatase inhibitors and methothrexate.
